(19) United States
(12) Patent Application Publication
(10) Pub. No.: US 2016/0380708 A1
(43) Pub. Date: Dec. 29, 2016

(54) SYSTEMS AND METHODS FOR RESOLVING INGESTIBLE EVENT MARKER (IEM) CONTENTION

(71) Applicant: Proteus Digital Health, Inc., Redwood City, CA (US)

(72) Inventors: Aditya Dua, San Jose, CA (US); Mark Zdeblick, Portola Valley, CA (US); Lawrence Arne, Palo Alto, CA (US); Jonathan Withrington, San Francisco, CA (US); William McAllister, Saratoga, CA (US); Alireza Shirvani, Menlo Park, CA (US); Jafar Shenasa, San Jose, CA (US); Jeremy Frank, San Francisco, CA (US); Patricia Johnson, Palo Alto, CA (US); Scott Andrews, Redwood City, CA (US); Robert Azevedo, Albany, CA (US); William A. Weeks, Santa Cruz, CA (US); Alberto Berstein, San Jose, CA (US); Yashar Behzadi, Anaheim, CA (US); Veeraperumanallu Muralidharan, Santa Clara, CA (US)

(21) Appl. No.: 15/190,741

(22) Filed: Jun. 23, 2016

Related U.S. Application Data
(60) Provisional application No. 62/185,374, filed on Jun. 26, 2015.

(51) Int. Cl. H04B 15/00; H04W 72/04; A61B 5/053; A61B 90/00; A61B 5/00; H04B 13/00; H04W 52/18

(52) U.S. Cl. CPC H04B 15/005; H04B 13/005; H04W 72/0446; H04W 72/0453; H04W 52/18; A61B 90/39; A61B 5/4842; A61B 5/4848; A61B 5/0028; A61B 5/002; A61B 5/0538; A61B 2562/0219

(57) ABSTRACT

Systems, methods, and apparatuses are presented for resolving any interference, noise, and/or collisions caused by two or more ingestible event markers (IEMs) transmitting simultaneously or concurrently. Methods include techniques from the perspective of the IEM for randomly varying signal transmission characteristics in order to avoid collisions with other concurrently transmitting IEMs. Methods also include techniques from the perspective of a receiver for receiving multiple transmission signals from multiple IEMs and for resolving any interference or signal collisions.

### CROSS-REFERENCE TO RELATED APPLICATION

This application claims the benefit under 35 USC §119(e) of U.S. Provisional Application No. 62/185,374, entitled SYSTEMS AND METHODS FOR RESOLVING INGESTIBLE EVENT MARKER (IEM) CONTENTION, filed on Jun. 26, 2015, the disclosure of which application is herein incorporated by reference.

### TECHNICAL FIELD

The subject matter disclosed herein generally relates to systems for detecting one or more events. In some embodiments, the present disclosures relate to systems and methods for resolving ingestible event marker (IEM) contention.

### BACKGROUND

Transbody communications are finding increasing use in medical applications. The term “transbody communications” generally refers to transmission of a signal from an in vivo location to a receiver location, e.g., a second in vivo location, a receiver location extracorporeally associated with the body, etc. These transbody communications may be accomplished through transmissions from one or more ingestible event markers (IEMs) that are ingested by a living being and activated after entering the body in order to signal the occurrence of an event. These communications, however, may be susceptible to errors. In particular, noisy transmission environments may distort and corrupt communication data. The noisy transmission environments may include instances where multiple communications from multiple in vivo transmitters are transmitting simultaneously. Additionally, communication devices may err in signal generation and measurement related to the communication data. As such, there is a continued need for accurate communications and error free data between multiple transmitters and at least one receiver configured to communicate with the in vivo transmitters.

### SUMMARY

In one embodiment, an ingestible event marker is provided. The ingestible event marker comprising: a partial power source comprising: a first material; and a second material electrically isolated from the first material, the first and second materials selected to provide a voltage potential difference as a result of the materials being in contact with a conductive liquid; a transmitter configured to transmit conductive signals through the conductive liquid; and a control device electrically coupled to the first and second materials and the transmitter and configured to: alter conductance between the first and second materials; encode signature information in a conductive transmission signal remotely detectable by a receiver, the signature information uniquely identifying the ingestible event marker; and encode a first random signal transmission characteristic, the first random signal transmission characteristic randomly altering transmission of the conductive transmission signal according to a first characteristic; provide a first instruction to transmit the conductive transmission signal including the first random signal transmission characteristic; encode a second random signal transmission characteristic, the second random signal transmission characteristic randomly altering transmission of the conductive transmission signal according to a second characteristic; and provide a second instruction to transmit the conductive transmission signal including the second random signal transmission characteristic.

Another embodiment provides the ingestible event marker, wherein providing the first instruction to transmit the transmission signal comprises altering the conductance between the first and second materials such that the magnitude of the current flow is varied to encode the signature information in the conductive transmission signal through the conductive liquid that is detectable by the receiver.

Another embodiment provides any combination of the ingestible event markers described above, wherein providing the second instruction to transmit the conductive transmission signal comprises instructing the transmitter to transmit conductive transmission signals encoded with the second instruction and detectable by the receiver.

Another embodiment provides of any combination of the ingestible event markers described above, wherein the first materials is an anode and the second material is a cathode.

Another embodiment provides any combination of the ingestible event markers described above, wherein: encoding the first random signal transmission characteristic comprises altering a first time for transmitting a first conductive transmission signal by a first randomized time increment; and encoding the second random signal transmission characteristic comprises altering a second time for transmitting a second conductive transmission signal by a second randomized time increment.

Another embodiment provides any combination of the ingestible event markers described above, wherein: encoding the first random signal transmission characteristic comprises altering a first time gap for transmitting a first conductive transmission signal between the first instruction and the second instruction by a first randomized time increment; and encoding the second random signal transmission characteristic comprises altering a second time gap for transmitting a second conductive transmission signal between the second instruction and a third instruction to transmit the transmission signal by a second randomized time increment.

Another embodiment provides any combination of the ingestible event markers described above, wherein: encoding the first random signal transmission characteristic comprises altering a frequency for transmitting the transmission signal by a first randomized frequency increment; and encoding the second random signal transmission characteristic comprises altering the frequency for transmitting the transmission signal by a second randomized frequency increment.

In one embodiment, an ingestible event marker is provided. The ingestible event marker comprising: a partial power source comprising: a first anode material; a first insulating material coupled to the first anode material; a second anode material coupled to the first insulating material and isolated from the first anode material; a first cathode material; a second insulating material coupled to the first cathode material; and a second cathode material coupled to the second insulating material and isolated from the first cathode material; the first and second anode materials both electrically isolated from both the first and second cathode materials; the first anode material and first cathode material selected to provide a voltage potential difference as a result of the first anode material and first cathode material being in contact with a conductive liquid; the first and second insulating materials selected to prevent a voltage potential difference as a result of the first and second insulating material being in contact with the conductive liquid; the second anode material and second cathode material selected to provide a voltage potential difference as a result of the second anode material and second cathode material being in contact with the conductive liquid; and a control device electrically coupled to the first and second anode materials and the first and second cathode materials, and configured to: alter conductance between the first anode material and the first cathode material to provide power to the ingestible event marker; encode signature information in a conductive transmission signal remotely detectable by a receiver, the signature information uniquely identifying the ingestible event marker; and provide a first instruction to transmit the conductive transmission signal.

Another embodiment provides the ingestible event marker described above, wherein the control device is further configured to: alter conductance between the second anode material and the second cathode material to provide power to the ingestible event marker after the first anode material and the first cathode material is depleted.

Another embodiment provides any combination of the ingestible event markers described above, wherein the control device is further configured to: encode the signature information in a second transmission signal remotely detectable by a receiver, and provide a second instruction to transmit the conductive transmission signal based on being the ingestible event marker being powered by the second anode material and the second cathode material.

Another embodiment provides any combination of the ingestible event markers described above, wherein the first anode material and the first cathode material comprise a first thickness layer, and the second anode material and the second cathode material comprise a second thickness layer.

Another embodiment provides any combination of the ingestible event markers described above, wherein the ingestible event marker is encapsulated by a pharmaceutical product.

Another embodiment provides any combination of the ingestible event markers described above, wherein the ingestible event marker is positioned within the center of the pharmaceutical product.

Another embodiment provides any combination of the ingestible event markers described above, wherein the ingestible event marker is positioned asymmetrically within of the pharmaceutical product.

In one embodiment a receiver is provided. The receiver comprising: a housing; a power source secured within the housing; a processing engine electrically coupled to the power source and secured within the housing; and at least two electrodes electrically coupled to the processing engine and secured to the perimeter of the housing such that the electrodes come into contact with a patient's skin; wherein the processing engine is configured to: detect a plurality of conductive transmission signals of Varying frequency in the form of a potential voltage difference between the at least two electrodes, each conductive transmission signal transmitted from a plurality of ingestible event markers transmitting concurrently, the plurality of ingestible event markers ingested by the patient; and decode each of the plurality of conductive transmission signals to identify each of the plurality of ingestible event markers.

Another embodiment provides the receiver described above, wherein detecting the plurality of conductive transmission signals comprises filtering a first conductive transmission signal of the plurality of transmission signals.

Another embodiment provides any combination of the receiver described above, wherein detecting the plurality of conductive transmission signals comprises determining an acoustic distance to each of the plurality of ingestible event markers based on measuring acoustic signals.

Another embodiment provides any combination of the receiver described above, wherein detecting the plurality of conductive transmission signals comprises determining a spatial distance to each of the plurality of ingestible event markers based on computing timing measurements of the transmission signals.

Another embodiment provides any combination of the receiver described above, wherein detecting the plurality of conductive transmission signals comprises randomly varying an interval to perform a “sniff” operation, the “sniff” operation configured to detect the presence of an ingestible event marker transmitting a transmission signal.

### BRIEF DESCRIPTION OF THE DRAWINGS

Some embodiments are illustrated by way of example and not limitation in the figures of the accompanying drawings.

FIG. 1 provides an example scenario of a patient ingesting multiple IEMs that give rise to the need to resolve signal contention between the various IEMs, according to some embodiments.

FIG. 2 shows several examples of IEMs, like those ingested in the scenario of FIG. 1.

FIG. 3A shows the system of an IEM in more detail, including a framework, two digestible materials and a control device, according to some embodiments.

FIG. 3B shows the control device of an IEM in more detail, including a control module, a clock, a memory, and input and output leads, according to some embodiments.

FIG. 3C shows a detail of an electronic circuit for a signal generation element, according to some embodiments.

FIG. 3D shows a circuit diagram showing details of a dipole electrode driver implemented using conventional CMOS driver circuits, according to some embodiments.

FIG. 3E shows a driver circuit for a monopole antenna that can be implemented in conventional CMOS integrated circuits, according to some embodiments.

FIG. 4 shows additional variants of an IEM configured to resolve signal contention between multiple IEMs transmitting concurrently, according to some embodiments.

FIG. 5 provides a block functional diagram of an integrated circuit component of a signal receiver configured to receive signals from multiple IEMs and to resolve any signal contentions, according to some embodiments.

### DETAILED DESCRIPTION

The following detailed description should be read with reference to the drawings, in which identical reference numbers refer to like elements throughout the different figures. The drawings, which are not necessarily to scale, depict selective embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. As used in this specification and the appended claims, the singular forms “a,” “an,” and “the” include plural referents unless the context clearly indicates otherwise.

Systems, methods, and apparatuses are presented for resolving signal contentions between two or more ingestible event markers (IEMs). An IEM may be used to identify and track an event, including for medical and non-medical purposes. Examples of medical applications where one may wish to note an event that is specific to a given individual include, but are not limited to, the onset of one or more physiological parameters of interest, including disease symptoms, the administration of a medication, etc. Examples of non-medical applications where one desires to note an event that is specific to a given individual include, but are not limited to: the ingestion of certain types of foods, e.g., for individuals on controlled diets, the commencement of an exercise regimen, etc. The IEM may be used with pharmaceutical product and the event that is indicated is when the product is taken or ingested. The term “ingested” or “ingest” or “ingesting” is understood to mean any introduction of the system internal to the body.

In general, ingesting includes simply placing the IEM in the mouth all the way to the descending colon. Thus, the term ingesting refers to any instant in time when the IEM is introduced to an environment that contains a electrically conductive fluid. Another example would be a situation when a non electrically conductive fluid is mixed with an conductive fluid. In such a situation the system would be present in the non conductive fluid and when the two fluids are mixed, the IEM comes into contact with the conductive fluid and the system is activated. Yet another example would be an instance when the presence of certain conductive fluids needed to be detected. In such instances, the presence of the IEM, which would be activated, within the conductive fluid could be detected and, hence, the presence of the respective fluid would be detected.

When multiple IEMs are ingested into the body, the signals transmitted by the multiple IEMs may come into conflict with one another. For example, a receiver configured to receive the transmission signals of the multiple IEMs may be unable to identify each individual signal correctly due to signal interference caused by the multiple IEMs transmitting at the same time. This is commonly referred to as “collision.” A collision is the situation that occurs when two or more devices attempt to send a signal along the same transmission channel at the same time. The colliding of the signals can result in garbled, and thus useless, messages.

Aspects of the present disclosures discuss multiple methods, systems and apparatuses for resolving any interference, noise, and/or collisions caused by two or more IEMs transmitting simultaneously or concurrently.

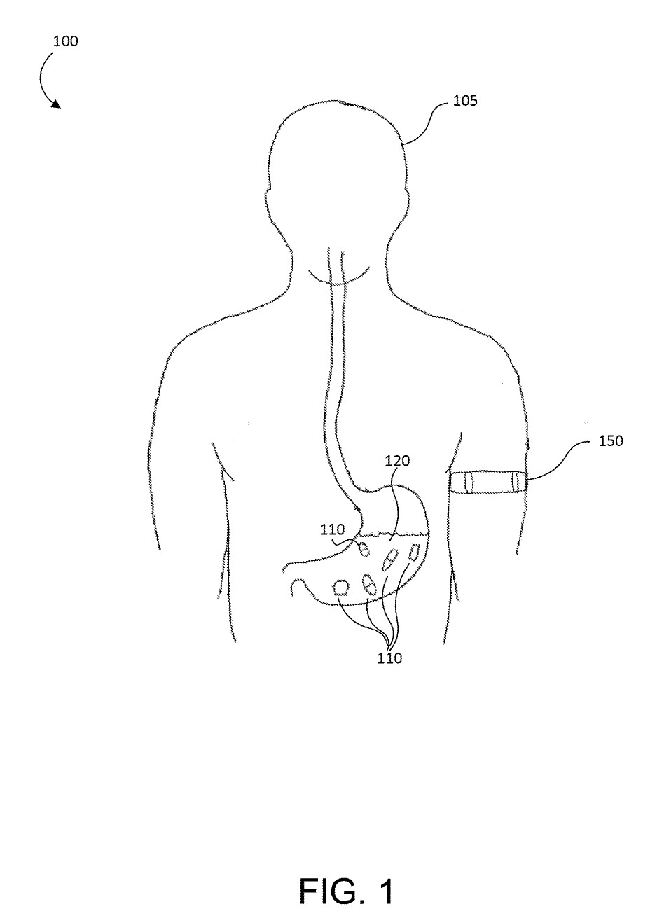

Referring to FIG. 1, illustration 100 provides an example scenario of a patient 105 ingesting multiple IEMs 110 that give rise to the need to resolve signal contention between the various IEMs 110, according to some embodiments. Here, the multiple IEMs 110 are configured as orally ingestible pharmaceutical formulations, each in the form of a pill or capsule. Upon ingestion of each IEM, the pill moves to the stomach. Upon reaching the stomach, each IEM 110 is in contact with stomach fluid 120 and undergoes a chemical reaction with the various materials in the stomach fluid 120, such as hydrochloric acid and other digestive agents. In general, an IEM 110 can be used in any environment where a conductive fluid is present or becomes present through mixing of two or more components that result in a conductive liquid.

Also shown is example receiver 150. As described further below, the IEMs 110 may be configured to transmit one or more signals after being activated through the conductive liquid (e.g., stomach fluid 120). The receiver 150 may be configured to receive the signals from the multiple IEMs 110. As described further below, the receiver 150 may receive the signals through one or more means, including, for example, radio frequency (RF) wireless transmission, physiological electrical means, and other transbody mediums. Here, the receiver 150 is represented as an arm band that may include electrodes attached to the skin of the patient 105. The receiver 150 is not limited to being placed on the patient's arm. For example, the receiver 150 may be designed in the form of a patch that sticks to the patient on his shoulder or hip. In other cases, the receiver 150 may not be attached to the body but may still be configured to receive RF signals or other wireless signals from the multiple IEMs 110. As another example, a wireless device, such as a smartphone or smartwatch, may have a software application installed that is configured to utilize the wireless receiver of the wireless device to act as the receiver 150.

As shown, the presence of the multiple IEMs 110 being ingested concurrently may allow all the IEMs 110 to transmit their designed signals at the same time. Thus, the signals may interfere with each other when a receiver is trying to identify each IEM. The receiver 150 may be configured to receive signals from multiple IEMs 110 simultaneously or concurrently through one or more of the various techniques for resolving IEM signal contention as described herein.

Example IEM Characteristics

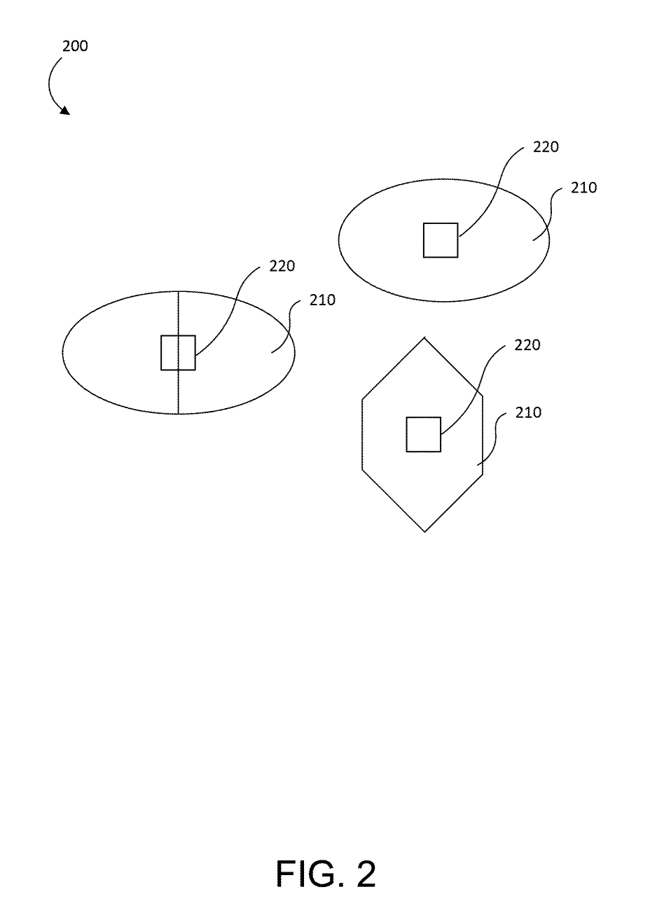

Referring to FIG. 2, illustration 200 shows several examples of IEMs, like those ingested in the illustration 100. The IEMs of the present disclosures are not limited by the shape or type. For example, the IEMs 110 can include a composition such as a pharmaceutical product 210 having an outer coating with accompanying medicine, and may be in the form of a capsule, a time-release oral dosage, a tablet, a gel cap, a sub-lingual tablet, or any oral dosage product. The pharmaceutical product 210 may be shaped in various ways, such as the oval capsules or the hexagonal form as shown. The pharmaceutical product 210 can be combined with a system 220 configured to be activated when in contact with a conductive fluid. The system 220 may be configured to transmit an encoded signal that may be received by a receiver, such as receiver 150, among other functions. In these examples, the pharmaceutical product 210 has the system 220 secured to the exterior using known methods of securing micro-devices to the exterior of pharmaceutical products. Examples of methods for securing the micro-device to the product is disclosed in U.S. Provisional Application No. 61/142,849 filed on Jan. 1, 2009 and entitled “HIGH-THROUGHPUT PRODUCTION OF INGESTIBLE EVENT MARKERS” and published as US patent application publication 2012/0011699 A1 as well as U.S. Provisional Application No. 61/177,611 filed on May 12, 2009 and entitled “INGESTIBLE EVENT MARKERS COMPRISING AN IDENTIFIER AND AN INGESTIBLE COMPONENT” and published as US patent application publication 2012/0059257 A1, of which the entire disclosure of each is incorporated herein by reference. Once ingested, the system 220 comes into contact with body liquids and the system 220 is activated. In some embodiments, the system 220 uses the voltage potential difference to power up and thereafter modulates conductance to create a unique and identifiable current signature. Upon activation, the system 220 controls the conductance and, hence, current flow to produce the current signature.

In certain embodiments, the system 220 employs a conductive near-field mode of communication in which the body itself is employed as a conductive medium. In such embodiments, the systems 220 include circuitry that, when freed from the composition upon disruption of the composition the circuitry comes into direct contact with the body and does not remain encapsulated or protected in some manner. In these embodiments, the signal is not a magnetic signal or high frequency (RF) signal, but is a near-field conductive signal transmitted using the body as a conductive medium.

In some embodiments, activation of the system 220 by being in contact with the conductive fluid may be intentionally delayed. In order to delay the activation of the system 220, the system 220 may be coated with a shielding material or protective layer, which may be part of the pharmaceutical product 210. The layer may be dissolved over a period of time, thereby allowing the system 220 to be activated when the product 210 has reached a target location.

In some embodiments, the pharmaceutical product 210 may simply be a capsule as a carrier for the system 220, and may not contain any product. Furthermore, the scope of the present disclosure is not limited by the shape or type of product 210. As shown, the product 210 has the system 220 positioned inside or secured to the interior of the product 210. In some embodiments, the system 220 is secured to the interior wall of the product 210. When the system 220 is positioned inside a gel capsule, then the content of the gel capsule is a non-conducting gel-liquid. On the other hand, if the content of the gel capsule is a conducting gel-liquid, then in alternative embodiments, the system 220 is coated with a protective cover to prevent unwanted activation by the gel capsule content. As another example, if the content of the capsule is a dry powder or microspheres, then the system 220 is positioned or placed within the capsule. As another example, if the product 210 is a tablet or hard pill, then the system 220 is held in place inside the tablet. Once ingested, the product 210 containing the system 220 is dissolved. The system 220 comes into contact with body liquids and the system 220 is activated. Depending on the product 210, the system 220 may be positioned in either a near-central or near-perimeter position depending on the desired activation delay between the time of initial ingestion and activation of the system 220. For example, a central position for the system 220 means that it will take longer for the system 220 to be in contact with the conductive liquid and, hence, it will take longer for the system 220 to be activated. Therefore, it will take longer for the occurrence of the event to be detected.

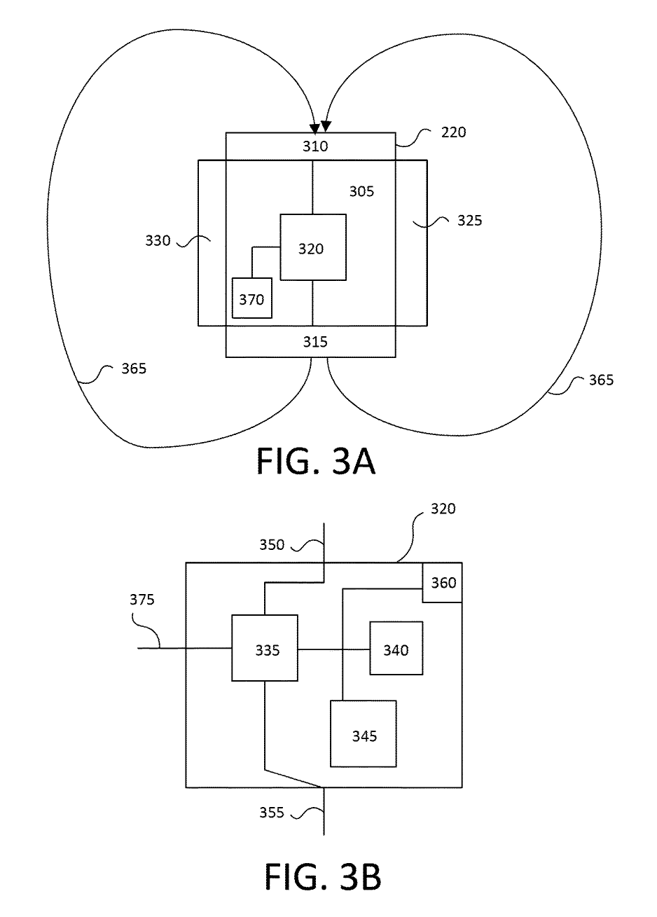

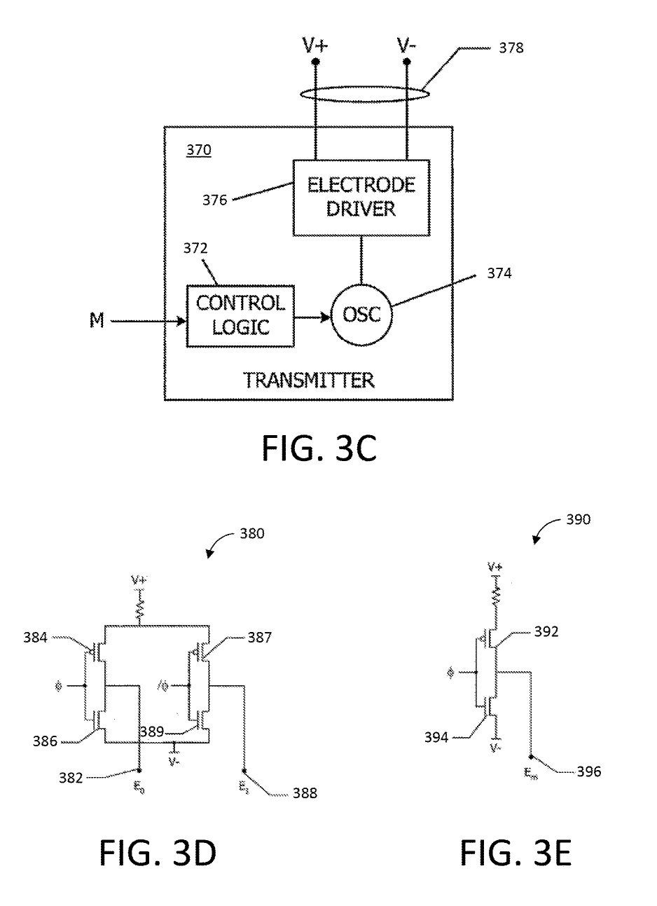

Referring now to FIG. 3A, in some embodiments, the system 220 is shown in more detail. Here, the system 220 can be used in association with any pharmaceutical product, such as product 210 as mentioned above, to determine when a patient 105 takes the pharmaceutical product. In general, the scope of the present disclosure is not limited by the environment and the product that is used with the system 220. For example, the system 220 may be placed within a capsule and the capsule is placed within the conductive liquid, e.g., through ingestion by the patient 105. The capsule would then dissolve over a period of time and release the system 220 into the conductive liquid.

Once in direct contact with the conductive fluid, e.g., stomach fluid 120, the system 220 is activated. The system 220 controls conductance to produce a unique current signature that is detected, thereby signifying that the pharmaceutical product has been taken. Here, the system 220 includes a framework 305. The framework 305 may serve as a chassis for the system 220 and multiple components are attached to, deposited upon, or secured to the framework 305. In this example of the system 220, a digestible material 310 is physically associated with the framework 305. The material 310 may be chemically deposited on, evaporated onto, secured to, or built-up on the framework all of which may be referred to herein as “deposited” with respect to the framework 305. In this example, the material 310 is deposited on one side of the framework 305. Examples of materials that can be used as material 310 include Cu or CuI. The material 310 may be deposited by physical vapor deposition, electrodeposition, or plasma deposition, among other protocols. In some embodiments, the material 310 may be from about 0.05 to about 500 μm thick, such as from about 5 to about 100 μm thick. The shape may be controlled by shadow mask deposition, or photolithography and etching. Additionally, even though only one region is shown for depositing the material, each system 220 may contain two or more electrically unique regions where the material 310 may be deposited, as desired.

At a different side, for example the opposite side as shown in FIG. 3A, another digestible material 315 is deposited, such that materials 310 and 315 are dissimilar. Although not shown, the different side selected may be the side next to the side selected for the material 310. In general, the term “different side” can mean any of the multiple sides that are different from the first selected side, and embodiments are not so limited. Furthermore, even though the shape of the system 220 is shown as a square, the shape may be any geometrically suitable shape, and embodiments are not so limited. Materials 310 and 315 are selected such that they produce a voltage potential difference when the system 220 is in contact with conductive liquid, such as body fluids. For example, materials for material 315 may include Mg, Zn, or other electronegative metals. In general, the materials 310 and 315 can be any pair of materials with different electrochemical potentials. As indicated above with respect to the material 310, the material 315 may be chemically deposited on, evaporated onto, secured to, or built-up on the framework 305. Also, an adhesion layer may be necessary to help the material 315 (as well as material 310 when needed) to adhere to the framework 305. Typical adhesion layers for the material 315 include Ti, TiW, Cr or similar material. Anode material and the adhesion layer may be deposited by physical vapor deposition, electrodeposition or plasma deposition. In some embodiments, the material 315 may be from about 0.05 to about 500 μm thick, such as from about 5 to about 100 μm thick. In general, the system 220 is not limited by the thickness of any of the materials nor by the type of process used to deposit or secure the materials to the framework 305.

Additionally, in the embodiments wherein the system 220 is used in-vivo, the materials 310 and 315 may be vitamins that can be absorbed. More specifically, the materials 310 and 315 can be made of any two materials appropriate for the environment in which the system 220 will be operating. For example, when used with an ingestible product, the materials 310 and 315 are any pair of materials with different electrochemical potentials that are ingestible. An illustrative example includes the instance when the system 220 is in contact with an ionic solution, such as stomach acids. Suitable materials are not restricted to metals, and in certain embodiments the paired materials are chosen from metals and non-metals, e.g., a pair made up of a metal (such as Mg) and a salt (such as CuCl or CuI). With respect to the active electrode materials, any pairing of substances—metals, salts, or intercalation compounds—with suitably different electrochemical potentials (voltage) and low interfacial resistance are suitable.

Materials and pairings of interest include, but are not limited to, those reported in Table 1 below. In one embodiment, one or both of the metals may be doped with a non-metal, e.g., to enhance the voltage potential created between the materials as they come into contact with a conductive liquid. Non-metals that may be used as doping agents in certain embodiments include, but are not limited to: sulfur, iodine and the like. In another embodiment, the materials are copper iodine (CuI) as the anode and magnesium (Mg) as the cathode. Embodiments of the present invention use electrode materials that are not harmful to the human body.

TABLE 1

| | Anode | Cathode |
|---|---|---|
| Metals | Magnesium, Zinc | |
| | Sodium (†), | |
| | Lithium (†) | |
| | Iron | |
| Salts | | Copper salts: iodide, chloride, bromide, |
| | | sulfate, formate, (other anions possible) |
| | | Fe³⁺ salts: e.g. orthophosphate, |
| | | pyrophosphate, (other anions possible) |
| | | Oxygen (††) on platinum, gold or other |
| | | catalytic surfaces |
| Intercalation | Graphite with Li, | Vanadium oxide |
| compounds | K, Ca, Na, Mg | Manganese oxide |

Thus, when the system 220 is in contact with the conductive liquid, a current path, starting from material 315 and ending at material 310, for example, is formed through the conductive liquid between materials 310 and 315. In some embodiments, a control device 320 is secured to the framework 305 and electrically coupled to the materials 310 and 315. The control device 320 includes electronic circuitry, for example control logic that is capable of controlling and altering the conductance between the materials 310 and 315.

The voltage potential created between the materials 310 and 315 provides the power for operating the system as well as produces the current flow through the conductive fluid and the system. In some embodiments, the system operates in direct current mode. In other cases, the system controls the direction of the current so that the direction of current is reversed in a cyclic manner, similar to alternating current. As the system reaches the conductive fluid or the electrolyte, where the fluid or electrolyte component is provided by a physiological fluid, e.g., stomach acid, the path for current flow between the materials 310 and 315 is completed external to the system 220; the current path through the system 220 is controlled by the control device 320. Completion of the current path allows for the current to flow and in turn a receiver, e.g. receiver 150, can detect the presence of the current and recognize that the system 220 has been activate and the desired event is occurring or has occurred.

In some embodiments, the two materials 310 and 315 are similar in function to the two electrodes needed for a direct current power source, such as a battery. The conductive liquid acts as the electrolyte needed to complete the power source. The completed power source described is defined by the physical chemical reaction between the materials 310 and 315 of the system 220 and the surrounding fluids of the body. The completed power source may be viewed as a power source that exploits reverse electrolysis in an ionic or a conduction solution such as gastric fluid, blood, or other bodily fluids and some tissues. Additionally, the environment may be something other than a body and the liquid may be any conductive liquid. For example, the conductive fluid may be salt water or a metallic based paint.

In certain embodiments, these two materials are shielded from the surrounding environment by an additional layer of material. Accordingly, when the shield is dissolved and the two dissimilar materials are exposed to the target site, a voltage potential is generated.

In certain embodiments, the complete power source or supply is one that is made up of active electrode materials, electrolytes, and inactive materials, such as current collectors, packaging, etc. The active materials are any pair of materials with different electrochemical potentials. Suitable materials are not restricted to metals, and in certain embodiments the paired materials are chosen from metals and non-metals, e.g., a pair made up of a metal (such as Mg) and a salt (such as CuI). With respect to the active electrode materials, any pairing of substances—metals, salts, or intercalation compounds—with suitably different electrochemical potentials (voltage) and low interfacial resistance are suitable.

A variety of different materials may be employed as the materials that form the electrodes. In certain embodiments, electrode materials are chosen to provide for a voltage upon contact with the target physiological site, e.g., the stomach, sufficient to drive the system of the identifier. In certain embodiments, the voltage provided by the electrode materials upon contact of the metals of the power source with the target physiological site is 0.001 V or higher, including 0.01 V or higher, such as 0.1 V or higher, e.g., 0.3 V or higher, including 0.5 volts or higher, and including 1.0 volts or higher, where in certain embodiments, the voltage ranges from about 0.001 to about 10 volts, such as from about 0.01 to about 10 V.

Still referring to FIG. 3A, the materials 310 and 315 provide the voltage potential to activate the control device 320. Once the control device 320 is activated or powered up, the control device 320 can alter conductance between the materials 310 and 315 in a unique manner. By altering the conductance between materials 310 and 315, the control device 320 is capable of controlling the magnitude of the current through the conductive liquid that surrounds the system 220. This produces a unique current signature that can be detected and measured by a receiver, e.g., receiver 150 which can be positioned internal or external to the body. In addition to controlling the magnitude of the current path between the materials, non-conducting materials, membrane, or “skirt” are used to increase the “length” of the current path and, hence, act to boost the conductance path, for example as disclosed in the U.S. patent application Ser. No. 12/238,345 titled, “In-Body Device with Virtual Dipole Signal Amplification” filed Sep. 25, 2008 and now U.S. Pat. No. 8,961,412, the entire content of which is incorporated herein by reference. Alternatively, throughout the disclosure herein, the terms “non-conducting material,” “membrane,” and “skirt” are interchangeably with the term “current path extender” without impacting the scope or the present embodiments and the claims herein. The skirt, shown in portion at 325 and 330, respectively, may be associated with, e.g., secured to, the framework 305. Various shapes and configurations for the skirt are contemplated as within the scope of the present invention. For example, the system 220 may be surrounded entirely or partially by the skirt and the skirt maybe positioned along a central axis of the system 220 or off-center relative to a central axis. Thus, the scope of the present invention as claimed herein is not limited by the shape or size of the skirt. Furthermore, in other embodiments, the materials 310 and 315 may be separated by one skirt that is positioned in any defined region between the materials 310 and 315.

In some embodiments, a transmitter 370 or signal generation component also may be embedded into the framework 305 and electrically coupled to the control device 320. Powered by the conductance between the materials 310 and 315, the control device 320 may be configured to transmit near-field conductive signals through the transmitter 370. The signals may include a representation of the current signature used to uniquely identify the activation of the system 220. In some embodiments, the transmitter 370 may transmit a second signature that can still uniquely identify the activation of the system 220. In some embodiments, the near-field conductive signals may be identified through transmission of a packet header, similar to packets employed in conventional wireless transmissions, and may follow known protocols, such as 802.11 protocols, although the near-field signals are conductively transmitted using the body as a conductive medium and are not RF wireless signals. In other cases, manufacturers of the IEM 110 and the receiver 150 may design different formats for wireless signals that are unique to transmission of IEMs, and embodiments are not so limited.

In some embodiments, at the surface of the material 310, there is chemical reaction between the material 310 and the surrounding conductive fluid such that mass is released into the conductive fluid. The term “mass” as used herein refers to protons and neutrons that form a substance. In one example, the material 310 is CuCl and when in contact with the conductive fluid, CuCl becomes Cu (solid) and Cl⁻ in solution. The flow of ions into the conduction fluid is depicted by the ion paths 365. In a similar manner, there is a chemical reaction between the material 315 and the surrounding conductive fluid and ions are captured by the material 315. The release of ions at the material 310 and capture of ion by the material 315 is collectively referred to as the ionic exchange. The rate of ionic exchange and, hence the ionic emission rate or flow, is controlled by the control device 320. The control device 320 can increase or decrease the rate of ion flow by altering the conductance, which alters the impedance, between the materials 310 and 315. Through controlling the ion exchange, the system 220 can encode information in the ionic exchange process. Thus, the system 220 uses ionic emission to encode information in the ionic exchange.

The control device 320 can vary the duration of a fixed ionic exchange rate or current flow magnitude while keeping the rate or magnitude near constant, similar to when the frequency is modulated and the amplitude is constant. Also, the control device 320 can vary the level of the ionic exchange rate or the magnitude of the current flow while keeping the duration near constant. Thus, using various combinations of changes in duration and altering the rate or magnitude, the control device 320 encodes information in the current flow or the ionic exchange. For example, the control device 320 may use, but is not limited to any of the following techniques namely, Binary Phase-Shift Keying (PSK), Frequency modulation, Amplitude modulation, on-off keying, and PSK with on-off keying.

Through altering the conductance in a specific manner, in some embodiments, the system 220 is capable of encoding information in the ionic exchange and the current signature. The ionic exchange or the current signature may be used to uniquely identify the specific system. Additionally, the system 220 may be capable of producing various different unique exchanges or signatures and, thus, provide additional information, according to some embodiments. For example, a second current signature based on a second conductance alteration pattern may be used to provide additional information, which information may be related to the physical environment. To further illustrate, a first current signature may be a very low current state that maintains an oscillator on the chip and a second current signature may be a current state at least a factor of ten higher than the current state associated with the first current signature.

In certain embodiments, the identifier system 220 emits a signal upon activation by a stimulus, e.g., by interrogation, upon contact with a target physiological location, etc. As such, the identifier system 220 may be an identifier that emits a signal when it contacts a target body (i.e., physiological) site. In addition or alternatively, the identifier system 220 may be an identifier that emits a signal when interrogated.

In some embodiments, depending on the needs of a particular application, the signal generated by the identifier system 220 may be a generic signal, e.g., a signal that merely identifies that the composition has contacted the target site, or a unique signal, e.g., a signal which in some way uniquely identifies that a particular composition from a group or plurality of different compositions in a batch has contacted a target physiological site. As such, the identifier system 220 may be one that, when employed in a batch of unit dosages, e.g., a batch of tablets, emits a signal which can be distinguished from the signal emitted by the identifier systems of any other unit dosage member of the batch. In yet other embodiments, the identifier system 220 emits a signal that uniquely identifies a given unit dosage, even from other identical unit dosages in a given batch. Accordingly, in certain embodiments the identifier system 220 emits a unique signal that distinguishes a given type of unit dosage from other types of unit dosages, e.g., a given medication from other types of medications. In certain embodiments, the identifier system 220 emits a unique signal that distinguishes a given unit dosage from other unit dosages of a defined population of unit dosages, e.g., a prescription, a batch or a lifetime production run of dosage formulations. In certain embodiments, the identifier system 220 emits a signal that is unique, i.e., distinguishable, from a signal emitted by any other dosage formulation ever produced, where such a signal may be viewed as a universally unique signal (e.g., analogous to a human fingerprint which is distinct from any other fingerprint of any other individual and therefore uniquely identifies an individual on a universal level). In one embodiment, the signal may either directly convey information about the composition, or provide an identifying code, which may be used to retrieve information about the composition from a database, i.e., a database linking identifying codes with compositions.

The identifier system 220 may be any component or device that is capable of generating a detectable signal following activation in response to a stimulus. In certain embodiments, the stimulus activates the identifier system 220 to emit a signal once the composition comes into contact with a physiological target site, e.g., as summarized herein. For example, a patient may ingest a pill that upon contact with the stomach fluids, generates a detectable signal. Depending on the embodiment, the target physiological site or location may vary, where representative target physiological sites of interest include, but are not limited to: a location in the gastrointestinal tract (such as the mouth, esophagus, stomach, small intestine, large intestine, etc.); another location inside the body, such as a parental location, vascular location, etc.; or a topical location; etc.

In certain embodiments the stimulus that activates the identifier system 220 is an interrogation signal, such as a scan or other type of interrogation. In these embodiments, the stimulus activates the identifier system 220, thereby emitting a signal which is then received and processed, e.g., to identify the composition in some manner.

In certain of these embodiments, the identifier system 220 may include a power source that transduces broadcast power and a signal generating element that modulates the amount of transduced power, such that a signal is not emitted from the identifier but instead the amount of broadcast power transduced by the identifier system 220 is detected and employed as the “signal.” Such embodiments are useful in a variety of applications, such as applications where the history of a given composition is of interest.

In certain embodiments, the identifier system 220 is dimensioned to be complexed with the active agent/pharmaceutically acceptable carrier component of the composition so as to produce a composition that can be readily administered to a subject in need thereof. As such, in certain embodiments, the identifier system 220 is dimensioned to have a width ranging from about 0.05 mm to about 1 mm, such as from about 0.1 mm to about 0.2 mm; a length ranging from about 0.05 mm to about 1 mm, such as from about 0.1 mm to about 0.2 mm and a height ranging from about 0.1 mm to about 1 mm, such as from about 0.05 mm to about 0.3 mm, including from about 0.1 mm to about 0.2 mm. In certain embodiments the identifier system 220 is 1 mm³ or smaller, such as 0.1 mm³ or smaller, including 0.2 mm³ or smaller. The identifier system 220 may take a variety of different configurations, such as but not limited to: a chip configuration, a cylinder configuration, a spherical configuration, a disc configuration, etc, where a particular configuration may be selected based on intended application, method of manufacture, etc.

The identifier system 220 may generate a variety of different types of signals, including but not limited to, RF, magnetic, conductive (near field), acoustic, etc.

As is known in the art (see, e.g., J. D. Jackson, Classical Electrodynamics, 2nd Edition, pp. 394-396 (1975)), the electric (E) and magnetic (B) fields for radiation of an oscillating electric dipole antenna with an angular frequency ω and corresponding wave number k (where k=ω/c, with c being the speed of light in the relevant medium) are given by the equations:

$$\begin{matrix}B = {k}^{2} ( n \times p ) \frac{{e}^{i kr}}{r} ( 1 - \frac{1}{i kr} ) ; \\ and & ( 1 ) \\ E = {k}^{2} ( n \times p ) \times n \frac{{e}^{i kr}}{r} + [ 3 n ( n \cdot p ) - p ] ( \frac{1}{{r}^{3}} - \frac{i k}{{r}^{2}} ) {e}^{i kr} . & ( 2 )\end{matrix}$$

where n is a unit vector in the direction from the center of the dipole source to a location x at a distance r from the source, and p is a space-integrated density of electric charge given by p=∫x′ρ(x′)d³x′.

As can be seen from Eqs. (1) and (2), in the “far field” region, where r>>λ, (where the wavelength λ=2π/k), the electric and magnetic fields are dominated by terms that decrease with distance as λ˜r. In this region, mutually perpendicular electric and magnetic fields feed off one another to propagate the signal through space. Where λ>>r, the l/r² (“induction”) terms in Eqs. (1) and (2) become significant, and where λ>>r, an additional quasi-electrostatic term that varies as l/r³ also becomes significant.

Conventional RF communication takes place at distances r˜λ to r>>λ. For instance, implantable medical devices such as pacemakers typically communicate in the 405-MHz frequency band, corresponding to wavelengths of 0.75 meters, somewhat smaller than the scale of a human body. As is known in the art, higher frequencies are advantageously not used because structures within the body begin to absorb radiation, leading to undesirable signal loss; substantially lower frequencies (longer wavelengths) are generally regarded as undesirable because much of the energy is redirected into the induction and/or quasi-static field components rather than the far-field component that can be sensed using conventional antennas. It should also be noted that RFID applications with a transponder and a base unit typically use wavelengths such that r˜λ. and generally rely on magnetic induction to transmit power from the transponder to the base unit. In certain embodiments, these RF signals may be employed.

In contrast to these approaches, certain embodiments of the present invention advantageously operate at wavelengths much larger than the human body (λ>>1 meter) to communicate information within the patient's body, e.g., as described in U.S. Provisional Application Ser. No. 60/713,680; the disclosure of which is herein incorporated by reference. For instance, in some embodiments, frequencies on the order of 100 kHz, corresponding to wavelengths of around 3 km (in air), are advantageously used. At distances r that are short as compared to the wavelength λ, the quasi-static electric field term in Eqs. (1) and (2) dominates, and thus the propagating signal is predominantly electrical rather than electromagnetic. Such signals readily propagate in a conductive medium such as the human body. For instance, at a frequency of 100 kHz and distances on the order of 1-2 meters, the quasi-static (l/r³) component of Eq. (2) is estimated to be on the order of 10⁶ times stronger than the far-field (l/r) component. Thus, long-wavelength signaling using near-field coupling is efficient. Further, because the signals are required to travel relatively short distances (typically 2 meters or less), detectable signals can be transmitted using very small antennas.

A wide range of frequencies may be used for transmission of signals. In some embodiments, the transmission frequency is within the “LF” band (low frequency, defined as 30-300 kHz) of the RF spectrum, below the frequency range of AM radio (around 500 to 1700 kHz). Within the LF band, the range from 160-190 kHz has been designated by the FCC for experimental use, with specified upper limits on external signal strength. In embodiments of the present invention where the signals are largely confined within the patient's body as described below, this experimental band can be used.

However, the disclosed embodiments are not limited to the 160-190 kHz band or to the LF (30-300 kHz band). Lower bands may also be used; for instance, in the VLF band (3-30 kHz, wavelengths of 10-100 km in air), signals can penetrate water to a distance of 10-40 meters. Since the electrical properties of the human body are similar to those of salt water, it is expected that signals in this band would also readily propagate through the body. Thus, any frequency band corresponding to a wavelength that is at least an order of magnitude larger than the human body—e.g., λ˜10 m or longer, or frequencies on the order of 30 MHz or below—can be used.

While there is no necessary lower limit on the frequency of signals used, several practical considerations may affect the choice of frequency. For instance, it is well known that the human body carries low-level oscillating signals induced by nearby AC-powered devices, which operate at 60 Hz (US) or similar frequencies in other parts of the world. To avoid interference caused by AC electrical power systems, frequencies near 60 Hz are advantageously not used. In addition, as is known in the art, longer wavelengths correlate with lower information transfer rates, and the information-transfer capacity at long wavelengths (e.g., below the 3 kHz-30 kHz VLF band) may be too small for the amount of information that is to be transferred in a particular system. Further, longer wavelengths generally require longer dipole antennas to produce a detectable signal, and at some point the antenna size may become a limiting factor in frequency selection.

According to some embodiments, given a suitable choice of frequency, a signal strong enough to travel to a receiver within the body can be generated using a very small antenna. For instance, 100 kHz signals generated by a dipole antenna just a few millimeters long can be propagated to a receiver antenna placed 1-2 meters away. This quasi-electrostatic transmission is believed to be aided by the fact that the implanted antenna is directly in contact with a conductive medium, for example, the patient's tissues. For purposes of analyzing electrical properties, human tissue can be approximated as an electrolyte solution with electrical properties comparable to those of salt water. Thus, as in an electrolyte bath, the quasi-electrostatic field created by an oscillating dipole antenna induces an oscillating current in the body. As a result of the inherent electrical resistivity of the body (comparable to salt water), the oscillating current creates oscillating potential variations within the body that can be sensed using a suitable receiver. (See, e.g., L. D. Landau et al. Electrodynamics of Continuous Media, Ch. 3 (1960)). Examples of suitable receivers include the leads of a pacemaker, which create a dipole with an axis of about 20 cm or any other implanted wires with length from 10-100 cm.

It should be noted that these currents are undesirable in the context of conventional RF communication, in which current flow in the near field leads to power loss in the far-field. In fact, many RF transmitters include devices designed to minimize near-field current leakage. In near-field transmitters of these embodiments of the present invention, maximizing such currents is desirable.

Further, for quasi-electrostatic signals, the patient's skin advantageously acts as a conductive barrier, confining the signals within the patient's body. This confines the signals within the body and also makes it difficult for stray external signals to penetrate the body and create noise or interference in the transmitted signals. Confinement of the signals can mitigate, to some extent, the l/r³ falloff of the near-field signal, further reducing power requirements. Such effects have been observed in the laboratory, e.g., in a salt water bath, in which the water/air interface acting as a conductive barrier. Similar effects have been observed in communicating with submarines via RF transmission in the ELF (3-30 Hz) and SLF (30-300 Hz) bands. These effects have also been observed in sonar communications; although sonar uses acoustic, rather than electrical or electromagnetic, fields to transmit information, the surface of the water acts as a conductive barrier for acoustic energy and mitigates the fall-off of signal intensity with distance.

As a result of these phenomena, a transmitter with a very small antenna and a small power source are sufficient to create a near-field signal that is detectable within the patient's body. For instance, the antenna can be formed by a pair of electrodes a few millimeters or less in length, spaced apart by a few millimeters, with oscillating voltages of opposite phase applied to create an oscillating electric dipole. Such antennas can be disposed almost anywhere within the body.

Further, in some embodiments, the frequency, transmitter antenna length, and receiver antenna length are selected such that only microwatts of power are required to produce a detectable signal, where conventional RF communication (e.g., at around 405 MHz) would require at least milliwatts. Accordingly, very compact power supplies that produce only small amounts of power can be used; examples are described in Section IV below.

As such, depending on the particular embodiment of interest, the frequency may range from about 0.1 Hz or lower to about 100 mHz or higher, e.g., from about 1 kHz to about 70 mHz, including from about 5 kHz to about 200 kHz.

In certain embodiment, the signal that is emitted by the identifier is an acoustic signal. In these embodiments, any convenient acoustic signal generation element may be present in the identifier, e.g., a piezoelectric element, etc.

The transmission time of the identifier may vary, where in certain embodiments the transmission time may range from about 0.1 μsec to about 4 hours or longer, such as from about 1 sec to about 4 hours. Depending on the given embodiment, the identifier may transmit a signal once or transmit a signal two or more times, such that the signal may be viewed as a redundant signal.

In certain embodiments, the identifier may be one that is programmable following manufacture, in the sense that the signal generated by the identifier may be determined after the identifier is produced, where the identifier may be field programmable, mass programmable, fuse programmable, and even reprogrammable. Such embodiments are of interest where uncoded identifiers are first produced and following incorporation into a composition are then coded to emit an identifying signal for that composition. Any convenient programming technology may be employed. In certain embodiments, the programming technology employed is RFID technology. RFID smart tag technology of interest that may be employed in the subject identifiers includes, but is not limited to: that described in U.S. Pat. Nos. 7,035,877; 7,035,818; 7,032,822; 7,031,946, as well as published application no. 20050131281, and the like, the disclosures of which are herein incorporated by reference. With RFID or other smart tag technology, a manufacturer/vendor may associate a unique ID code with a given identifier, even after the identifier has been incorporated into the composition. In certain embodiments, each individual or entity involved in the handling of the composition prior to use may introduce information into the identifier, e.g., in the form of programming with respect to the signal emitted by the identifier, e.g., as described in U.S. Pat. No. 7,031,946 the disclosure of which is herein incorporated by reference.

The identifier of certain embodiments includes a memory element, where the memory element may vary with respect to its capacity. In certain embodiments, the memory element has a capacity ranging from about 1 bit to 1 gigabyte or more, such as 1 bit to 1 megabyte, including from about 1 bit to about 128 bit. The particular capacity employed may vary depending on the application, e.g., whether the signal is a generic signal or coded signal, and where the signal may or may not be annotated with some additional information, e.g., name of active agent, etc.

Identifier systems 220 embodiments have: (a) an activation component and (b) a signal generation component, where the signal generation component is activated by the activation component to produce an identifying signal, e.g., as described above.

Referring to FIG. 3B, a block diagram representation of the control device 320 is shown. The device 320 includes a control module 335, a counter or clock 340, and a memory 345. Additionally, the device 320 is shown to include a sensor module 360. In some embodiments, the sensor module 360 is located outside the control device 320 while still attached to the framework 305 and electrically coupled to the control device 320. The control module 335 has an input 350 electrically coupled to the material 310 and an output 355 electrically coupled to the material 315. In addition, the control module 335 may have an input 375 to the transmitter 370. The control module 335, the clock 340, the memory 345, and the sensor module 360 also have power inputs (some not shown). The power for each of these components is supplied by the voltage potential produced by the chemical reaction between materials 310 and 315 and the conductive fluid, when the system 220 is in contact with the conductive fluid. The control module 335 controls the conductance through logic that alters the overall impedance of the system 220. The control module 335 is electrically coupled to the clock 340. The clock 340 provides a clock cycle to the control module 335. Based upon the programmed characteristics of the control module 335, when a set number of clock cycles have passed, the control module 335 alters the conductance characteristics between materials 310 and 315. This cycle is repeated and thereby the control device 320 produces a unique current signature characteristic. The control module 335 is also electrically coupled to the memory 345. Both the clock 340 and the memory 345 are powered by the voltage potential created between the materials 310 and 315.

In some embodiments, the control module 335 is also electrically coupled to and in communication with the sensor module 360. Here, the sensor module 360 is part of the control device 320, while in other cases, the sensor module 360 is separate from the control module 360 but is still electrically coupled to the control module 360. Any component of the system 220 may be functionally or structurally moved, combined, or repositioned, and embodiments are not so limited. Thus, it is possible to have one single structure, for example a processor, which is designed to perform the functions of all of the following modules: the control module 335, the clock 340, the memory 345, and the sensor module 360. In addition, each of these functional components may be located in independent structures that are linked electrically and able to communicate, and embodiments are not so limited.

In some embodiments, the sensor module 360 may include any of the following sensors: temperature, pressure, pH level, and conductivity. In some embodiments, the sensor module 360 gathers information from the environment and communicates the analog information to the control module 335. The control module then converts the analog information to digital information and the digital information is encoded in the current flow or the rate of the transfer of mass that produces the ionic flow. In another embodiment, the sensor module 360 gathers information from the environment and converts the analog information to digital information and then communicates the digital information to control module 335.

FIG. 3C shows a detail of an electronic circuit for a signal generation element, according to some embodiments. In some embodiments, the transmitter 370 is a signal generation element. The signal generation element 370 employs an electric dipole or electric monopole antenna to transmit signals. FIG. 3C illustrates a dipole antenna. Oscillator 374 provides driving signals (φ and an inverted signal denoted herein as /φ) to an electrode driver 376. FIG. 3D shows a circuit diagram showing details of a dipole electrode driver 380 implemented using conventional CMOS driver circuits, according to some embodiments. Electrode 382 is driven to a potential E₀ by transistors 384, 386 in response to driving signal φ while electrode 388 is driven to a potential E₁ by transistors 387, 389 in response to inverted driving signal /φ. Since driving signals φ and /φ oscillate with opposite phase, potentials E₀ and E₁ also oscillate with opposite phase. It will be appreciated that driver 380 and all other electronic circuits described herein can be implemented using sub-micron CMOS processing technologies known in the art; thus, the size of the circuitry is not a limiting factor on the size of a remote device.

In some embodiments, a monopole antenna can be substituted for the dipole antenna of FIG. 3C. FIG. 3E shows a driver circuit 390 for a monopole antenna that can be implemented in conventional CMOS integrated circuits, according to some embodiments. This antenna driver 390 is generally similar to one half of the driver circuit of FIG. 3D, with driver transistors 392, 394 driving a single electrode 396 to a potential Eₘ response to driving signal φ.

In either the dipole or monopole case, the driver circuit is powered by a potential difference (ΔV) between terminals V+ and V−. This potential difference, which can be constant or variable, as desired.

Turning back to FIG. 3C, there is shown a block diagram of a transmitter signal generation element 370 for an identifier system 220 according to an embodiment. In this embodiment, the signal generation element 370 receives a signal M from the activation component of the identifier system 220 which activates the signal generation element 370 to produce and emit a signal. Signal generation element 370 includes control logic 372, an oscillator 374, an electrode driver 376, and an antenna 378 (in this instance, a pair of electrodes operated as an electric dipole antenna). In operation, the oscillator 374 generates an oscillating signal (waveform) in response to signals from the control logic 372. The signals from the control logic 372 can start or stop the oscillator and in some embodiments can also shape one or more aspects of the oscillatory signal such as amplitude, frequency, and/or phase. The oscillator 374 provides the waveform to the electrode driver 376, which drives current or voltage on the antenna 378 to transmit a signal into the conductive medium of body tissues or fluids.

Depending on a given embodiment, the signal may or may not be modulated. For example, in certain embodiments the frequency of the signal may be held constant. In yet other embodiments, the signal may be modulated in some manner, e.g., via carrier based modulate schemes, ultra-wide band (or time domain based) modulation schemes, etc.

Referring again to FIG.3C, in some embodiments, the oscillator 374 operates at a constant frequency. The receipt of a constant-frequency signal in and of itself can provide useful information, e.g., that a remote device is present and operational. In some embodiments, the oscillator 374 modulates its signal to encode additional information.

Information can be encoded in various ways, generally by modulating (varying) some property of the transmitted signal, such as frequency, amplitude, phase, or any combination thereof. Modulation techniques known in the art may be employed.

In general, information can be transmitted using analog or digital techniques. “Analog techniques” refers generally to instances in which the modulated property is varied in different degrees, with the degree of variation being correlated to a value representing the information to be transmitted. For instance, suppose that the signal generation element 370 is transmitting a signal. The oscillator 374 can be designed to operate over some range of frequencies. “Digital techniques” refers generally to instances in which the information to be transmitted is represented as a sequence of binary digits (bits), and the signal is modulated based on the bit stream. For instance, suppose again that the transmitter signal generation element 370 is transmitting a signal using digital techniques. The oscillator 374 can be designed to operate at least two different frequencies, with one frequency corresponding to bit value 0 and another frequency corresponding to bit value 1. In various embodiments, either analog techniques, digital, techniques, or a combination thereof can be used to transmit information. In addition, various types of modulation may be implemented. As described in more detail below, the frequencies used to represent the bit value 0 or the bit value 1 may vary to avoid transmission collisions with other identifier systems attempting to transmit.

In one embodiment, frequency modulation is used. The oscillator 370 can be a voltage-controlled oscillator (VCO), an oscillator circuit in which the oscillation frequency depends on an applied voltage. The control logic 372 supplies an appropriate voltage (e.g., reflecting the value of the measurement data, M), and the frequency of the signal indicates the value of the data. In another embodiment, amplitude modulation is used; for instance, the amplitude of the driving signals φ and /φ can be varied, or the positive and negative rails of the driver circuit (e.g., V+ and V−) can be varied to control the amplitude. In another embodiment, phase modulation is used. For instance, in digital signal transmission, one phase corresponds to bit value 0, an opposite phase corresponds to bit value 1, and the phase shifts represent transitions. The oscillator 374 can include a switch circuit that either directly connects oru cross-connects the driving signals φ and /φ to the inputs of a driver circuit. Combinations of frequency modulation, amplitude modulation, and/or phase modulation may also be used as desired.

In some embodiments, the transmitter signal generation element 370 may transmit a “packet” that includes a unique identifier for the identifier, which in turn is for the composition with which the identifier is associated. The unique identifier may also provide information from the remote device (e.g., the identity of the active agent (i.e., annotation information)). Other techniques for distinguishing different signals may also be used, including: operating different transmitters in different frequency bands, allowing each transmitter to be identified by its frequency and/or configuring different transmitters to transmit at different (and known) times, allowing the transmitter to be identified by when it transmits.

Example Techniques for Modifying IEMs to Resolve IEM Transmission Contention

Based on the above descriptions of example foundational implementations for an IEM, the IEM, e.g., IEM 110, may be modified in various ways to resolve conflicts where multiple IEMs may transmit their unique signatures or other relevant communications concurrently.

For example, in some embodiments, the control module 335 may be configured to start transmitting the unique signature of the IEM after a random period of time upon being activated. The control module 235 may begin transmitting the unique signature of the IEM after waiting a random amount of time based on a pseudorandom seed obtained from the memory 345. The timing may be adjusted based on the clock cycles from the clock 340. Thus, in instances where multiple pharmaceutical products containing IEMs are ingested at the same time, e.g., patient 105 swallows multiple pills at once, the actual start times of the transmission of the unique signatures may be staggered or varied randomly, based on each random seed in each IEM. A receiver configured to receive the signals from each of the IEMs, e.g., receiver 150, may accept and identify a first unique signature, and thereafter filter out that signal if the IEM continues to transmit, in order to receive and identify the signature of a second IEM, and so forth.

In some embodiments, the control module 335 may be configured to employ a frequency hopping functionality. The frequency hopping functionality may be associated with the specific communications channel(s), frequency hopping protocol, etc. As such, various aspects may utilize one or more frequency hopping protocols. For example, the receiver may search the designated range of frequencies, e.g., two or more different frequencies, in which the transmission could fall. In some embodiments, the two or more frequencies that may be hopped to may be programmed at the factory level, while in other cases the control module 335 may randomly generate two or more frequencies to hop from upon activation. In these cases, the receiver 150 may be configured to scan a broad spectrum of frequencies in order to detect signal transmission. When a single proper decode is achieved, the in vivo transmitter 370 has accomplished its mission of communicating its digital information payload to the receiver.

In some embodiments, the control module 335 may employ duty cycle modulation, wherein the transmitter 370 need not transmit all the time. If two IEMs are not transmitting simultaneously, they will not interfere with each other. For example, if two IEMs are used which have low duty cycles, such as broadcasting 10% of the time and off 90% of the time, then probabilistically there is only a 20% chance that the signals will overlap with each other. In this manner, collisions may be avoided.

For example, suppose a first IEM is transmitting only on 10% of the time. A second IEM is also transmitting only on 10% of the time. Of course, there is some probability that they will transmit simultaneously. However, that probability can be controlled by changing the duty cycle and the frequency spread, in some embodiments. As a result, if these two transmit periods are slightly different, they will come in and out of interference with each other. The overlap can be controlled, however, by dithering the duty cycle and the frequency spread. Dithering the duty cycle and/or the frequency spread may be based on applying a pseudorandom seed that modifies when an IEM is instructed to transmit, for example. In this manner, otherwise occurring collisions may be avoided.

In some embodiments, the transmission periods of an IEM may be varied compared to other IEMs and/or the transmission period of the same IEM for its given signature may be randomly varied. In the former instance, a first IEM may have a slightly shorter period than a second IEM, for example. Even though the transmitters begin broadcasting at the same time, after some number of transmissions, the transmitters come out of alignment with each other. As a result, they are now distinct from one another and otherwise occurring collisions may be avoided. In the latter instance, even IEM's that have substantially the same transmission period can be distinguished if the length of at least one of the transmission periods is randomly varied. This may be accomplished by the control module 335 appending to a transmission packet a random amount of filler data, the random amount based on a pseudorandom number or generator supplied by the memory 345, for example.

In some embodiments, a similar effect can be obtained by having a spread of oscillator frequencies. In practice, the silicon oscillators used for these transmitters have a spread of a few percent in frequency. A 1% difference in frequency means that after a 100 transmissions, two oscillators that began in phase with each other are no longer in phase with each other. Various aspects may be based on frequency distribution or the frequencies can also be programmed to be explicitly different, e.g., to have some range of periods. Noise dithering a voltage controlled oscillator frequency can also create this frequency spread.

In some embodiments, the retry period between transmitting the signature is randomized or at least is modified by some random time period. For example, a first IEM may broadcast and then waits some random period of time before broadcasting again. The first IEM then waits another random period of time before broadcasting again, and so forth. A second IEM may begin broadcasting at the same time. However, in this case it waits a random time before the next transmission, and waits another random time before the next transmission and so forth. In this way, the probability that two transmitters broadcast simultaneously can be controlled by affecting the standard deviation of the retry periods. This approach can be based on a pseudo-random sequence that is preprogrammed into the memory 345. It can also be based on a real physical random number generator (thermal noise), or on the serial number on the system 220. Every IEM may have a unique serial number, some of the lower bits of the serial number can be used to program this randomization time, either directly or by using a linear shift register.

In some embodiments, transbody transmission techniques use spread spectrum transmission to modulate the transmit message. This approach can be direct spread spectrum or frequency hopping spread spectrum. As an example, any of the code division multiple access (CDMA) techniques developed for cell phones that allow for multitudes of cell phones to broadcast on the same frequency without interference can be employed in these examples. These variations can also be based on any of the well known codes in spread spectrum, such as Gold Codes or Kasami codes.

In another embodiment, the transmission technique may employ blind source separation (BSS) to resolve the problem IEM transmission contention among in IEM signals. BSS is a technique to decode signals generated by multiple sources at the same time. The objective is to resolve each signal based on the output of multiple receivers in different locations. Hence, each of the IEM signals can be resolved by collecting data from different receivers placed in different locations on the body using the BSS technique. The power of BSS lies in the fact that it can be applied to non-linear channels as well. Recently, BSS has been applied to biomedical signals like EEG, ECG, EMG, MMG, etc.

The challenge to be addressed is approached probabilistically. A code is selected such that there are sufficiently many that the probability of two transmitters having the same code broadcasting at the same time is sufficiently small. This approach ties into the idea of using a beacon to find the carrier frequency because spread spectrum transmissions in general do not have a well defined carrier frequency. In some embodiments, the codes may be preprogrammed into each IEM upon manufacture. In other cases, the codes may be programmed specifically for each patient, where the number of possible IEM transmission contentions may be known based on each patient's pharmaceutical regimen. The receiver 150 associated with each patient may also be calibrated to look for each of these preprogrammed codes.

In calculations, it is shown that duty cycle works very well for two or three IEMs operating simultaneously. However, if the duty cycle method does not account for many IEMs, adding retransmit randomization may bolster the chances of successful multiple transmissions. In addition, spread spectrum is one approach of interest.

In some embodiments, it is also useful to increase the total time allowed for transmitting the IEM's signature. Reserving or conserving power may allow an IEM to have more chances of successfully transmitting its signature when there are multiple transmission conflicts. Reducing the duty cycle may help conserve battery life, for example, and thereby further increase the chances that the IEM may successfully transmit its signature and other information.

In some embodiments, the transmitter 370 may be implemented or programmed as a transceiver, configured to transmit and receive other signals from other IEMs. The transceiver 370 can listen for a quiet channel, for example, waiting until it hears nothing transmitting and then transmit. The spread spectrum approach is quantifiable, depending on how many distinct codes are used. When the Kasami set of codes are used there are 32,000 distinct codes. In this case, the probability of having two IEMs transmit on the same code is 1/(32,000)². That probability goes up geometrically with the number of transmitters. Even doing nothing to select transmitters that have distinct codes, and relying on the randomization of code selection, it supports tens, if not hundreds, of IEMs transmitting concurrently.

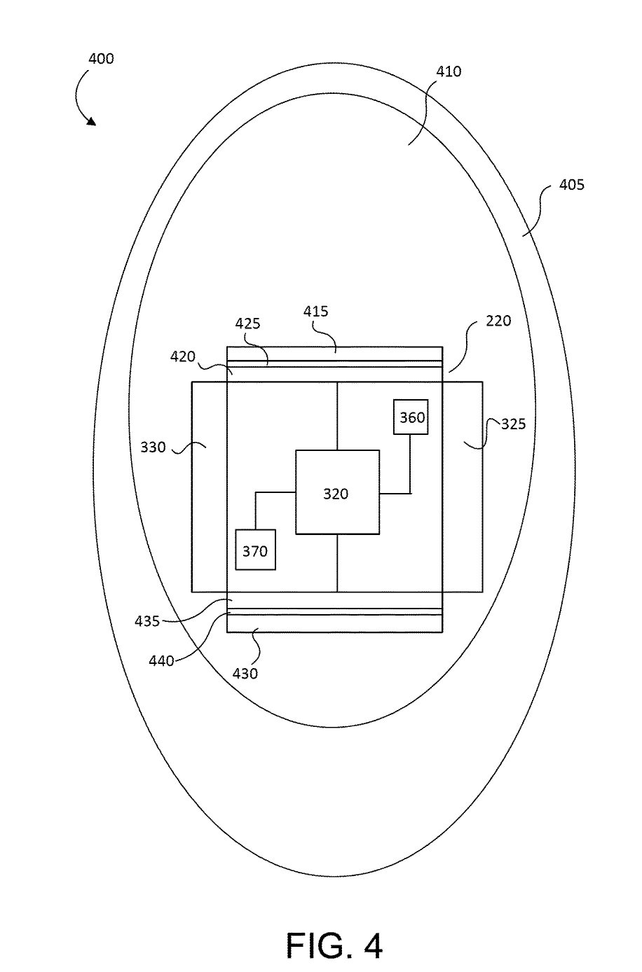

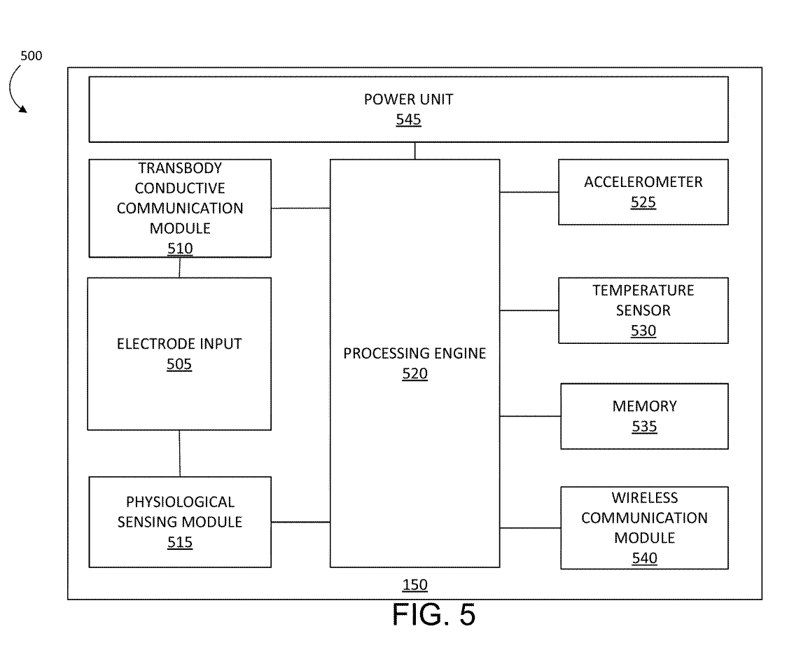

Referring to FIG. 4, illustration 400 shows additional variants of an IEM configured to resolve signal contention between multiple IEMs transmitting concurrently, according to some embodiments. As shown, in some embodiments, the system 220 is encased in a pharmaceutical product 405 having an oval shape and positioned substantially in the center of the product 405. In other cases, the system 220 is encased in a pharmaceutical product 410 having an oval shaped but positioned substantially to one end of the product 410, as shown. Thus, illustration 400 provides two possibilities for positioning of the system 220, to demonstrate that the positioning with in a product, e.g., a pharmaceutical product, may alter the timing for when the system 220 may be activated. For example, when the system 220 is encased in the pharmaceutical product 405, the product 405 may dissolve evenly around the system 220, such that the system 220 may be activated after all or nearly all of the product 405 has dissolved. In contrast, if the system 220 is encased in the product 410 and positioned substantially to one side, as shown, then the system 220 may be activated at a different timing, when just a portion of the product 410 has dissolved. Thus, if each IEM comprised of a product 405 and 410 were ingested at the same time by a patient, one of the IEM's may start transmitting its signature and other information earlier than the other.

In general, in some embodiments, the amount of a product coating encasing the system 220 can be varied to change the timings for when the system 220 may be activated. As another example, the type of the coating or type of material comprising the pharmaceutical product may be varied, such that the dissolving rates of each of the pharmaceutical products may vary. This also may allow the timings for when the system 220 encased inside to vary upon activation. As another example, the physical properties of the pharmaceutical product may be varied such that the rates of dissolving can vary depending on where the product has traveled within the body. For example, some materials may be more resistant to stomach acid, while others may be more resistant to saliva. Varying these physical properties also may change the start times of transmission.

In some embodiments, the system 220 may also include multiple layers of materials used to activate the control device 320. For example, as shown, instead of a single layer of material 310, the system 220 in illustration 400 includes two digestible layers 415 and 420, with a digestible insulation layer 425 in between. In some embodiments, the digestible layers 415 and 420 may be composed of the same material, while in other cases the materials may be different. The insulation layer 425 may be composed of a nonconducting layer that is still digestible but that prevents activation of the control device 320 when in contact with the conductive fluid. In some embodiments, the thickness of the digestible layers 415 and 420 may vary, such that the periods of activation of the control device 320 may subsequently vary. In other cases, the type of digestible material used for materials 415 and 420 may allow for different rates of dissolution, such that the periods of activation of the control device 320 may vary. Similarly, the system 220 also may include multiple layers of the second digestible material used to complete the circuit when in contact with the conductive fluid. That is, instead of a single layer of material 315, two digestible layers 430, 435 are included, with a digestible insulation layer 440 in between. Similar properties to the layers 415, 420, and 425, may apply to the different layers 430, 435, 440.

Due to the variation supplied by the varying properties of the materials 415, 420, 425, 430, 435, 440, the control device 320 may be activated at two distinct time periods. This may allow for an intentional break or pause between transmitting the signature or other information associated with the IEM. Thus, if the thickness or other physical properties of the digestible materials are varied, the timings of signal transmission may also be varied, thereby increasing the probability that any conflicting transmissions may be resolved.

Example Receiver Characteristics

Referring to FIG. 5, illustration 500 provides a block functional diagram of an integrated circuit component of a signal receiver configured to receive signals from multiple IEMs and to resolve any signal contentions, according to some embodiments. An example of the receiver in illustration 500 may include the receiver 150. In FIG. 5, receiver 150 includes electrode input 505. Electrically coupled to the electrode input 505 are a transbody conductive communication module 510 and a physiological sensing module 515, which are described more, below. In some embodiments, transbody conductive communication module 510 is implemented as a high frequency (HF) signal chain and physiological sensing module 515 is implemented as a low frequency (LF) signal chain. Also shown are CMOS temperature sensing module 530 (for detecting ambient temperature) and a 3-axis accelerometer 525. Receiver 150 in this example also includes a processing engine 520 (for example, a microcontroller and digital signal processor), non-volatile memory 535 (for data storage) and wireless communication module 540 configured to conduct data transmission to another device, for example in a data upload action or to receive RF signals, e.g. from wireless signals transmitted from transmitter 370. The receiver 150 may also include a power unit 545 configured to supply power to the various components and modules of the receiver 150.

As used herein, the transbody conductive communication module 510 is a functional module that is configured to receive a conductively transmitted signal, such as a signal emitted by an IEM 110. In some instances, the signal which the transbody conductive communication module is configured to receive is an encoded signal, which in some cases is a signal has been modulated in some manner (for example using a protocol such as binary phase shift keying (BPSK), frequency shift keying (FSK), amplitude shift keying (ASK), etc.). In such instances, the receivers and transbody conductive communication module 510 thereof are configured to decode a received encoded signal, such as an encoded signature emitted by the IEM 110. The receiver 150 may be configured to decode the encoded signal in a low signal to noise ratio (SNR) environment, e.g., where there may be substantial noise in addition to the signal of interest, e.g., an environment having an SNR of 7.7 dB or less. The receiver 150 may be further configured to decode the encoded signal with substantially no error. In certain aspects, the signal receiver has a high coding gain, e.g., a coding gain ranging from 6 dB to 12 dB, such as a coding gain ranging from 8 dB to 10 dB, including a coding gain of 9 dB. The signal receivers of aspects of the invention can decode encoded signals with substantially no error, e.g., with 10% error or less.

In those aspects where the received signal is encoded, such as where the received signal is an encoded IEM signal, the transbody conductive communication module 510 may be configured to process the received signal with at least one demodulation protocol, where the transbody conductive communication module 510 may be configured to process the received signal with two or more, three or more, four or more, etc., different demodulation protocols, as desired. When two or more different demodulation protocols are employed to process a given encoded signal, the protocols may be run simultaneously or sequentially, as desired. The received signal may be processed using any convenient demodulation protocol. Demodulation protocols of interest include, but are not limited to: Costas Loop demodulation; coherent demodulation; accurate, low overhead iterative demodulation; incoherent demodulation; and differential coherent demodulation.

In addition, the receiver 150 may include one or more distinct physiological sensing modules, e.g., physiological sensing module 515. As used herein, a physiological sensing module describes a capability or functionality of sensing one or more physiological parameters or biomarkers of interest, such as, but not limited to: cardia-data, including heart rate, electrocardiogram (ECG), and the like; respiration rate, temperature; pressure; chemical composition of fluid, e.g., analyte detection in blood, fluid state, blood flow rate, accelerometer motion data, etc. Where the receiver 150 has physiological parameter or biomarker sensing capability, the number of distinct parameters or biomarkers that the signal receiver may sense may vary, e.g., one or more, two or more, three or more, four or more, five or more, ten or more, etc. The term “biomarker” refers to an anatomic, physiologic, biochemical, or molecular parameter associated with the presence and severity of a health state, such as a specific disease state. In some embodiments, the receiver 150 may accomplish one or more of these sensing functions using a signal receiving element of the device, such as by using electrodes 505 for signal receiving and sensing applications, or the receiver may include one or more distinct sensing elements, such as micro-needles, that are different from the signal receiving element. The number of distinct sensing elements that may be present on the (or at least coupled to the) signal receiver 150 may vary, and may be one or more, two or more, three or more, four or more, five or more, ten or more, etc.

In some embodiments, as previously mentioned, the receiver 150 may also include one or more accelerometer modules, e.g., accelerometer 525. An accelerometer module is a module which is configured to obtain accelerometer data and, if desired, additionally perform one or more of processing the data in some way, storing the data and retransmitting the data. The accelerometer module may be employed by the receiver 150 to derive a number of different metrics, including but not limited to: data regarding patient activity, mean activity, patient position and angle, activity type, such as walking, sitting, resting (where this data may be obtained with a 3-axis accelerometer); and then save the obtained data. Of interest are both analog accelerometers and digital accelerometers.

In some embodiments, as previously mentioned, the receiver 150 may include an environmental functional module, e.g., temperature sensor 530. Environmental functional modules are modules that are configured to or acquire data related to the environment of the receiver, e.g., the environmental conditions, whether the receiver is connected to a skin surface, etc. For example, the environmental functional module may be configured to obtain receiver ambient temperature data. The environmental functional module may be configured to determine electrode connection, e.g., by impedance measurement. The environmental functional module may be configured to determine battery voltage. The above specific functions of the environmental functional module are merely illustrative and are not limiting.

The receiver 150 may be configured to handle received data in various ways. For example, the receiver 150 simply retransmits the data to an external device (e.g., using conventional RF communication via wireless communication module 540). In other aspects, the receiver processes the received data to determine whether to take some action such as operating an effector that is under its control, activating a visible or audible alarm, transmitting a control signal to an effector located elsewhere in the body, or the like. In some embodiments, the receiver 150 stores the received data for subsequent retransmission to an external device or for use in processing of subsequent data (e.g., detecting a change in some parameter over time). The receivers may perform any combination of these and/or other operations using received data.

In some embodiments, the data that are recorded on the data storage element, e.g. memory 535, includes at least one of, if not all of, time, date, and an identifier (e.g., global unique serial no.) of each IEM administered to a patient, where the identifier may be the common name of the composition or a coded version thereof. The data recorded on the data storage element of the receiver may further include medical record information of the subject with which the receiver is associated, e.g., identifying information, such as but not limited to: name, age, treatment record, etc. In certain aspects, the data of interest include hemodynamic measurements. In certain aspects, the data of interest include cardiac tissue properties. In certain aspects, the data of interest include pressure or volume measurements, temperature, activity, respiration rate, pH, etc.

Receivers may include a variety of different types of power sources which provide operating power to the device in some manner. The nature of the power unit 545 may vary. In some instances, the power unit 545 may include a battery. When present, the battery may be a onetime use battery or a rechargeable battery. For rechargeable batteries, the battery may be recharged using any convenient protocol. Of interest is a protocol that results in multi-tasking of elements of the receiver. For example, the receiver 150 may include one or more electrodes which are used for a variety of functions, such as receiving conductively transmitted signals, sensing physiological data, etc. The one or more electrodes, when present, may also be employed as power receivers which may be employed for recharging the rechargeable battery. Alternatively, the power unit 545 may be configured to receive a power signal, e.g., where the power unit 545 comprises a coil which can impart power to the device when an appropriate magnetic field is applied to the receiver. In yet other instances, the receiver 150 may include a body-powered power unit 545, such as that described in U.S. patent application Ser. No. 11/385,986 now UA Patent No. 7729768, the disclosure of which is herein incorporated by reference.

In some embodiments, the receiver 150 may be configured to control when certain states are assumed by the receiver 150, e.g., in order to minimize device power usage. For example, the processing engine 520 may implement a duty cycle for data collection based on time of day, or patient activity, or other events, where the implemented duty cycle may be based on a signal factor or multiple factors. For example, the processing engine 520 may cause the receiver 150 to obtain patient activity data (for example by an accelerometer module) when the patient is moving around and not when the patient is at rest.

In some embodiments, the receiver 150 may “wake up” periodically, and at low energy consumption, to perform a “sniff function” via, for example, the processing engine 520. The term “sniff function” generally refers to a short, low-power function to determine if a transmitter, e.g., an IEM, is present. If a transmitter signal is detected by the sniff function, the receiver 150 may transition to a higher power communication decode mode. If a transmitter signal is not present, the receiver 150 may return, e.g., immediately return, to sleep mode. In this manner, energy is conserved during relatively long periods when a transmitter signal is not present, while highpower capabilities remain available for efficient decode mode operations during the relatively few periods when a transmit signal is present. Several modes, and combination thereof, may be available for operating the sniff circuit. By matching the needs of a particular system to the sniff circuit configuration, an optimized system may be achieved.

Example Techniques for Modifying Receivers to Resolve IEM Transmission Contention

Based on the above descriptions of example foundational implementations for a receiver configured to receive IEM signals, the receiver, e.g., receiver 150, may be modified in various ways to resolve conflicts where multiple IEMs may transmit their unique signatures or other relevant communications concurrently.

For example, the receiver 150 may be configured to resolve signal transmission contention by filtering one or more frequencies or signals. By using frequency-selective filtering, a first IEM broadcasting at a first frequency can be distinguished from a second IEM broadcasting at a second frequency, even if they are transmitting simultaneously. In some embodiments, the receiver 150 may be preprogrammed to know which frequencies to monitor in order to detect the presence of an IEM signal. This may be based on knowledge of a patient's pharmaceutical regimen, for example, where the number and type of pills containing IEMs has been prescribed to the patient.

As an example, suppose a first IEM is broadcasting at a first frequency, and a second IEM is broadcasting at a second frequency. The receiver 150 may employ two band pass filters at the wireless communication module 540. In other cases, the receiver 150 may employ the two band pass filters at the transbody conductive communication module 510, which is configured to detect signals through the body at various frequencies. Band pass filter 1 is sensitive to the first frequency, while band pass filter 2 is sensitive to the second frequency. Once signals from the first and second IEMs, respectively, get through their respective band pass filters, the signals go to demodulators. In some embodiments, these demodulators can be implemented as separate analog circuits or in the digital domain. In this manner, collisions may be avoided.

In some embodiments, the receiver 150 may be configured to resolve IEM signals transmitting concurrently by examining other physiological properties of the patient during ingestion and digestion. For example, the physiological sensing module 515 may be configured to detect changes in the body of the patient, such as blood flow to the stomach, or changes in the energy level of the patient based on the type of material being digested in the pharmaceutical products. In some cases, certain materials that are digested and attached and activated with the conductive fluid may exhibit unique physiological properties that may be detectable by the physiological sensing module 515. In this way, the presence of multiple IEMs may be resolved.

In some embodiments, the receiver 150 may be configured to resolve IEM signals transmitting concurrently by conducting acoustic detection techniques similar to radar. For example, each of the ingested IEMs may have certain physical properties that, when an acoustic signal is directed to the patient's stomach, a distance can be measured based on its round-trip travel time in order to identify how many unique IEMs are present in the patient's stomach. These example acoustic techniques may be performed by the transbody conductive communication module 510, or the physiological sensing module 515, via the electrode inputs 505.

In some embodiments, the receiver 150 may be configured to resolve IEM signals transmitting concurrently by conducting signal timing measurements similar to beamforming. For example, an IEM may be configured to transmit a wireless signal having a time signature. When received by the receiver 150 at the wireless communication module 540, timing measurements may be obtained. Standard trilateration techniques may be employed to compute a distance to the IEM. Similarly, multiple IEM's may be identified in this way to determine how many IEM's are present in the patient's stomach.

In some embodiments, the receiver 150 may be configured to vary the “sniff” timing for when the receiver 150 attempts to detect the presence of a transmitter, e.g., an IEM. For example, the regular sniff timing of the receiver 150 may be modified by a randomized seed stored in the memory 535. Assuming then, for example, that each of the IEMs transmit at regular intervals, the receiver 150 may wake up to start catching the beginning of a transmission at different times. This may increase the probability that the receiver 150 catches the beginning of a transmission of a different IEM each time, thereby resolving any collisions.

In some embodiments, the receiver 150 may be configured to selectively receive a signal in a quiet part of a given spectrum. The wireless communication module 540 of the receiver 150 may be programmed to that frequency band in the quiet part of the given spectrum. An IEM may be programmed to periodically broadcast in that frequency band.

In some embodiments, the receiver 150 may be configured to record the entirety of a broad spectrum of frequencies, in order to record the transmission of many signals at once. Then, through postprocessing, performed either at the processing engine 520, or at a more powerful computer that receives the recorded raw data from the receiver 150, more sophisticated signal processing algorithms may be performed to identify the unique transmission signatures of each of the broadcasting IEMs.

In certain applications, it is useful to combine the different techniques mentioned herein. By example, when there is a long duty cycle, spread spectrum transmission can be particularly valuable. In this case, the probability of a collision happening is the probability of the long duty cycle times the probability of the spread spectrum. There are no restrictions on combining techniques.

Unless specifically stated otherwise, discussions herein using words such as “processing,” “computing,” “calculating,” “determining,” “presenting,” “displaying,” or the like may refer to actions or processes of a machine 1300 (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The present disclosure is illustrative and not limiting. Further modifications will be apparent to one skilled in the art in light of this disclosure and are intended to fall within the scope of the appended claims.

What is claimed is:

1. An ingestible event marker comprising:
a partial power source comprising:
a first material; and
a second material electrically isolated from the first material,
the first and second materials selected to provide a voltage potential difference as a result of the materials being in contact with a conductive liquid;
a transmitter configured to transmit conductive signals through the conductive liquid; and
a control device electrically coupled to the first and second materials and the transmitter and configured to:
alter conductance between the first and second materials;
encode signature information in a conductive transmission signal remotely detectable by a receiver, the signature information uniquely identifying the ingestible event marker; and
encode a first random signal transmission characteristic, the first random signal transmission characteristic randomly altering transmission of the conductive transmission signal according to a first characteristic;
provide a first instruction to transmit the conductive transmission signal including the first random signal transmission characteristic;
encode a second random signal transmission characteristic, the second random signal transmission characteristic randomly altering transmission of the conductive transmission signal according to a second characteristic; and
provide a second instruction to transmit the conductive transmission signal including the second random signal transmission characteristic.

2. The ingestible event marker of claim 1, wherein providing the first instruction to transmit the transmission signal comprises altering the conductance between the first and second materials such that the magnitude of the current flow is varied to encode the signature information in the conductive transmission signal through the conductive liquid that is detectable by the receiver.

3. The ingestible event marker of claim 2, wherein providing the second instruction to transmit the conductive transmission signal comprises instructing the transmitter to transmit conductive transmission signals encoded with the second instruction and detectable by the receiver.

4. The ingestible event marker of claim 1, wherein the first materials is an anode and the second material is a cathode.

5. The ingestible event marker of claim 1, wherein:
encoding the first random signal transmission characteristic comprises altering a first time for transmitting a first conductive transmission signal by a first randomized time increment; and
encoding the second random signal transmission characteristic comprises altering a second time for transmitting a second conductive transmission signal by a second randomized time increment.

6. The ingestible event marker of claim 1, wherein:
encoding the first random signal transmission characteristic comprises altering a first time gap for transmitting a first conductive transmission signal between the first instruction and the second instruction by a first randomized time increment; and
encoding the second random signal transmission characteristic comprises altering a second time gap for transmitting a second conductive transmission signal between the second instruction and a third instruction to transmit the transmission signal by a second randomized time increment.

7. The ingestible event marker of claim 1, wherein:
encoding the first random signal transmission characteristic comprises altering a frequency for transmitting the transmission signal by a first randomized frequency increment; and
encoding the second random signal transmission characteristic comprises altering the frequency for transmitting the transmission signal by a second randomized frequency increment.

8. An ingestible event marker comprising:
a partial power source comprising:
a first anode material;
a first insulating material coupled to the first anode material;
a second anode material coupled to the first insulating material and isolated from the first anode material;
a first cathode material;
a second insulating material coupled to the first cathode material; and
a second cathode material coupled to the second insulating material and isolated from the first cathode material;
the first and second anode materials both electrically isolated from both the first and second cathode materials;
the first anode material and first cathode material selected to provide a voltage potential difference as a result of the first anode material and first cathode material being in contact with a conductive liquid;
the first and second insulating materials selected to prevent a voltage potential difference as a result of the first and second insulating material being in contact with the conductive liquid;
the second anode material and second cathode material selected to provide a voltage potential difference as a result of the second anode material and second cathode material being in contact with the conductive liquid; and
a control device electrically coupled to the first and second anode materials and the first and second cathode materials, and configured to:
alter conductance between the first anode material and the first cathode material to provide power to the ingestible event marker;
encode signature information in a conductive transmission signal remotely detectable by a receiver, the signature information uniquely identifying the ingestible event marker; and
provide a first instruction to transmit the conductive transmission signal.

9. The ingestible event marker of claim 8, wherein the control device is further configured to:
alter conductance between the second anode material and the second cathode material to provide power to the ingestible event marker after the first anode material and the first cathode material is depleted.

10. The ingestible event marker of claim 9, wherein the control device is further configured to:
encode the signature information in a second transmission signal remotely detectable by a receiver, and
provide a second instruction to transmit the conductive transmission signal based on being the ingestible event marker being powered by the second anode material and the second cathode material.

11. The ingestible event marker of claim 8, wherein the first anode material and the first cathode material comprise a first thickness layer, and the second anode material and the second cathode material comprise a second thickness layer.

12. The ingestible event marker of claim 8, wherein the ingestible event marker is encapsulated by a pharmaceutical product.

13. The ingestible event marker of claim 12, wherein the ingestible event marker is positioned within the center of the pharmaceutical product.

14. The ingestible event marker of claim 12, wherein the ingestible event marker is positioned asymmetrically within of the pharmaceutical product.

15. A receiver comprising:
a housing;
a power source secured within the housing;
a processing engine electrically coupled to the power source and secured within the housing; and
at least two electrodes electrically coupled to the processing engine and secured to the perimeter of the housing such that the electrodes come into contact with a patient's skin;
wherein the processing engine is configured to:
detect a plurality of conductive transmission signals of Varying frequency in the form of a potential voltage difference between the at least two electrodes, each conductive transmission signal transmitted from a plurality of ingestible event markers transmitting concurrently, the plurality of ingestible event markers ingested by the patient; and
decode each of the plurality of conductive transmission signals to identify each of the plurality of ingestible event markers.

16. The receiver of claim 15, wherein detecting the plurality of conductive transmission signals comprises filtering a first conductive transmission signal of the plurality of transmission signals.

17. The receiver of claim 15, wherein detecting the plurality of conductive transmission signals comprises determining an acoustic distance to each of the plurality of ingestible event markers based on measuring acoustic signals.

18. The receiver of claim 15, wherein detecting the plurality of conductive transmission signals comprises determining a spatial distance to each of the plurality of ingestible event markers based on computing timing measurements of the transmission signals.

19. The receiver of claim 15, wherein detecting the plurality of conductive transmission signals comprises randomly varying an interval to perform a “sniff” operation, the “sniff” operation configured to detect the presence of an ingestible event marker transmitting a transmission signal.
